(19) United States
(12) Patent Application Publication
(10) Pub. No.: US 2015/0297088 A1
(43) Pub. Date: Oct. 22, 2015

(54) DISTRIBUTED PRESSURE SENSING SYSTEM FOR A MEDICAL DEVICE

(71) Applicant: Northeastern University, Boston, MA (US)

(72) Inventors: Casey R. Birmingham, New Brunswick, NJ (US); Scott Cooper, Morganville, NJ (US); Robert Hertig, Scituate, MA (US); Saeed Sokhanvar, Cambridge, MA (US)

(73) Assignee: Northeastern University, Boston, MA (US)

(21) Appl. No.: 14/362,297

(22) PCT Filed: Dec. 5, 2012
(86) PCT No.: PCT/US2012/067933

§ 371 Date: Jun. 2, 2014

Related U.S. Application Data
(60) Provisional application No. 61/567,065, filed on Dec. 5, 2011.

(51) Int. Cl. A61B 5/00

(52) U.S. Cl. CPC A61B 5/0084; A61B 5/4222

(57) ABSTRACT

The present disclosure describes a device and method for detecting distributed pressures along a medical device. The device includes an optical fiber that is helically wound around the flexible shaft of the medical device. Responsive to microbends caused by the application of a pressure to the optical fiber, attenuation occurs as light propagates down the optical fiber. The device detects the light attenuation and calculates the pressure exerted on the device. Accordingly, a physician can ensure pressure induced by the medical device does not surpass clinically safe levels.

### CROSS-REFERENCE TO RELATED PATENT APPLICATIONS

This application claims priority from Provisional U.S. Patent Application 61/567,065, filed Dec. 5, 2011, incorporated herein by reference in its entirety.

### BACKGROUND OF THE DISCLOSURE

Endoscopy is a minimally invasive medical procedure using an endoscope. The endoscope allows a physician to visually examine internal organs of the patient through a small incision in the patient. The device also provides suction, water dispensing, and surgical functions. During endoscopic operations it is possible to perforate an internal lining or organ of the patient. Perforations can be caused by several factors during a procedure, one of which is “looping.” Looping occurs when the rate at which the endoscope's flexible shaft advances is greater than the speed at which the tip of the endoscope advanced into the patient. When this occurs the flexible shaft can loop upon itself. The loop can exert pressure on the wall of a patient's lumen that is sufficient to cause a perforation. Looping is the leading cause of perforations during a colonoscopy.

### SUMMARY OF THE DISCLOSURE

The present disclosure describes a device and method for measuring the pressure a medical device, such as an endoscope, applies to an internal organ or lumen of a patient. The device includes an optical fiber that is helically wound around the flexible shaft of the medical device. The shaft also includes a number of small ridges, which deform the optical fiber when a pressure is applied to the device. As light propagating down the optical fiber encounters a microbend a portion of the light will scatter and be lost to the environment. The light attenuation can be measured and used to calculate the radius of the microbend and subsequently the pressure that caused the microbend. This system can then be used to warn physicians before a perforation occurs.

According to aspect of the disclosure, a pressure sensor for a medical device includes an elongated shaft having a first end, a second end, and a plurality of frets spaced a set distance apart along a substantial portion the elongated shaft. The device also includes a first optical fiber having an inlet and an outlet and running along a substantial length of the elongated shaft. A first light source is configured to project light into the inlet of the first optical fiber, and a first sensor is positioned to detect light emitted from the outlet of the first optical fiber.

In some implementations, the first light source and the first sensor are positioned near the first end of the elongated shaft and connected by the first optical fiber. In other implementations, the first optical fibers run non-tangentially over the frets. In yet other implementations, the device further includes a processor configured to calculate a pressure exerted on the elongated shaft.

In some implementations, the first optical fiber is helically wound around the elongated shaft, and the pitch of the first helically wound optical fibers is four times the set distance between adjacent frets.

In yet other implementations, the first light source is configured to project light of a first wavelength and second wavelength. The first wavelength and second wavelength are selected to attenuate at different rates when the first optical fiber is bent a set amount. In some implementations, the first optical fiber is multi-modal.

In other implementations, a second optical fiber is helically wrapped along the elongated shaft substantially parallel to the first optical fiber, and the second optical fiber is connected to a second sensor and a second light source emitting a second wavelength of light. In some implementations, the elongated shaft is part of a catheter, an endoscope, or a colonoscope.

According to another aspect of the disclosure, a method for measuring a pressure along a medical device includes wrapping a first optical fiber along an elongated shaft of the medical device, wherein a plurality of frets are spaced a set distance apart along substantial portion of the elongated shaft. The method also includes projecting, by a light source, a first wavelength of light into the first optical fiber, wherein the first wavelength of light has a first intensity. Responsive to projecting the light, the method includes detecting, by a sensor, a second intensity of the first wavelength of light when the first wavelength of light exits the optical fiber. The method also includes determining, by a processor, a pressure along the elongated shaft of the medical device by comparing the first intensity to the second intensity.

In some implementations, the method also includes projecting, by the light source, a second wavelength of light into the first optical fiber, wherein the second wavelength of light has a third intensity; and detecting, by the sensor, a fourth intensity of the second wavelength of light when the second wavelength of light exits the optical fiber. Furthermore, in some implementations, the method includes determining, by the processor, a distribution of the pressure along the elongated shaft of the medical device by comparing a first difference between the first intensity and second intensity to a second difference between the third intensity and fourth intensity.

In some implementations, the first wavelength and second wavelength are selected to attenuate at different rates when the first optical fiber is bent a set amount. In other implementations, the method includes wrapping a second optical fiber along the elongated shaft of the medical device; projecting, by a second light source, a second wavelength of light into the second optical fiber, wherein the second wavelength of light has a third intensity; and detecting, by a second sensor, a fourth intensity when the second wavelength of light exits the second optical fiber.

In yet other implementations, the method includes determining, by the processor, a distribution of the pressure along the elongated shaft of the medical device by comparing a first difference between the first intensity and second intensity to a second difference between the third intensity and fourth intensity.

In some implementations, the method includes warning a user if the pressure exceeds a set threshold. In other implementations, the method further includes helically wrapping the first optical fiber around the length of the elongated shaft and non-tangentially over the frets. In some of these implementations, the pitch of the helically wound optical fiber is four times the set distance between adjacent frets.

### BRIEF DESCRIPTION OF THE DRAWINGS

The skilled artisan will understand that the figures, described herein, are for illustration purposes only. It is to be understood that in some instances various aspects of the described implementations may be shown exaggerated or enlarged to facilitate an understanding of the described implementations. In the drawings, like reference characters generally refer to like features, functionally similar and/or structurally similar elements throughout the various drawings. The drawings are not necessarily to scale, emphasis instead being placed upon illustrating the principles of the teachings. The drawings are not intended to limit the scope of the present teachings in any way. The system and method may be better understood from the following illustrative description with reference to the following drawings in which:

FIG. 1 is a schematic diagram of one embodiment of a device for sensing pressure distributed pressures along a medical device, in accordance with an implementation of the present disclosure;

FIGS. 2A-C are detailed illustrations of different embodiments of possible optical fiber wrapping configurations of a device similar to the device of FIG. 1 in accordance with an implementation of the present disclosure;

FIG. 3 is a flow chart of one embodiment of a method for sensing distributed pressures using a device similar to the device of FIG. 1 in accordance with an implementation of the present invention;

FIG. 4 is an illustration of several possible endoscopic complications that may lead to bowel perforations;

FIG. 5A is a graph illustrating the relationship between bend radius and light attenuation of a device similar to the device of FIG. 1 in accordance with an implementation of the present disclosure;

FIG. 5B is a graph illustrating the theoretical relationship between pressure and light attenuation of a device similar to the device of FIG. 1 in accordance with an implementation of the present disclosure; and

FIG. 5C is a graph illustrating the experimental relationship between pressure and light attenuation of a device similar to the device of FIG. 1 in accordance with an implementation of the present disclosure.

### DETAILED DESCRIPTION

The following description in conjunction with the above-reference drawings sets forth a variety of implementations for exemplary purposes, which are in no way intended to limit the scope of the described methods or systems. Those having skill in the relevant art can modify the described methods and systems in various ways without departing from the broadest scope of the described methods and systems. Thus, the scope of the methods and systems described herein should not be limited by any of the exemplary implementations and should be defined in accordance with the accompanying claims and their equivalents.

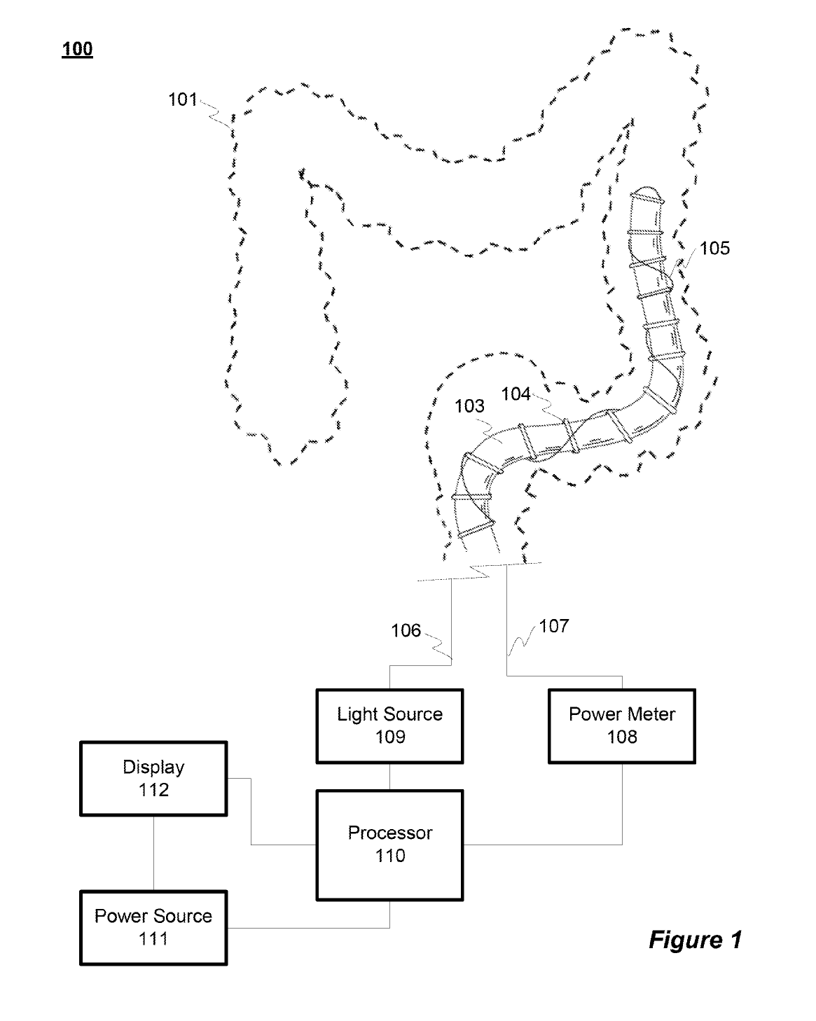

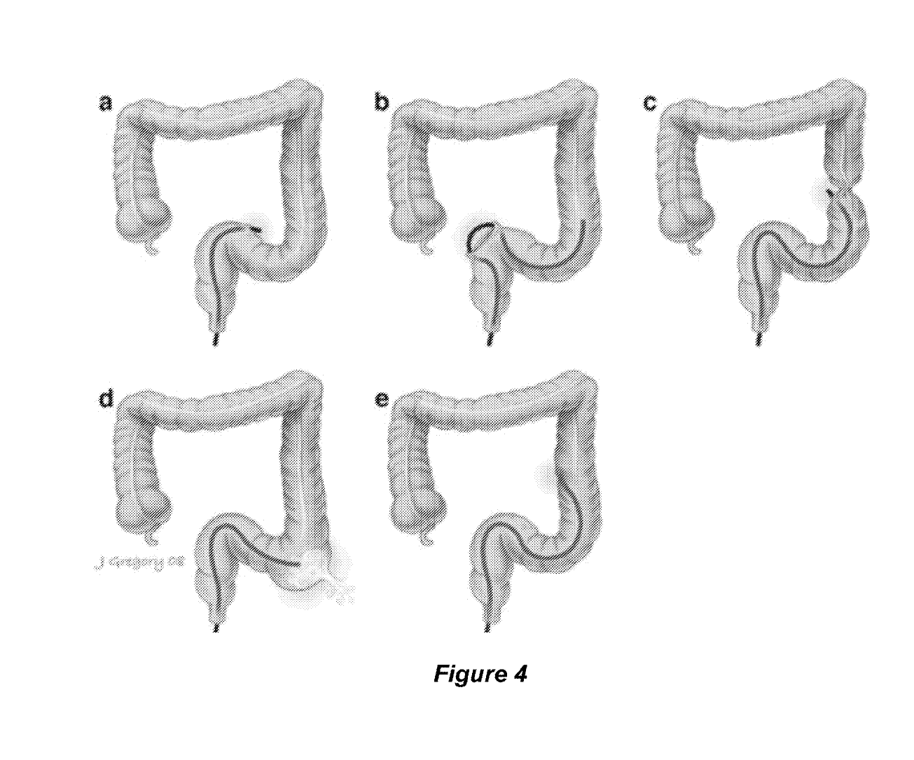

FIG. 1 is a schematic diagram of an embodiment of a system for detecting pressure along a medical device. More specifically, the device 100 may be incorporated into a medical device to warn a physician when the medical device may perforate an internal lumen or organ of a patient. For example, the device 100 may warn a physician when a colonoscope is exerting pressures capable of perforating a patient's colon. As a brief overview, FIGS. 4A-E illustrate a number of common bowel perforations induced by colonoscopes. These perforations can include perforations by the tip of the colonoscope (FIG. 4A, FIG. 4C, and FIG. 4E), over inflation of the bowels (FIG. 4D), and the most common perforation, “looping” (FIG. 4B).

In a brief overview, the device 100 may include a flexible or inflexible elongated shaft 103. A number of frets 104 may be spaced along the length of the shaft. A optical fiber 105, having an inlet portion 106 and an outlet portion 107 may be wrapped around the length of the shaft. The inlet portion 106 of the optical fiber is connected to a light source 109, and the outlet portion 107 of the optical fiber is connected to a power meter 108. A processor 110 may control the device 100 and provide output to a display 112. Finally, the components of the device 100 receive power from a power source 111.

Still referring to device 100 of FIG. 1, and in more detail, the device 100 may include an elongated shaft. The shaft 103 of the medical device 100 may be any elongated shaft of a medical device. In other implementations, the shaft 103 may be a sleeve or other configuration, which is not permanently attached to the shaft of a medical device. In some implementations, the device 100 may monitor the pressure a medical device exerts on the internal organs of patient. In the non-limiting example of FIG. 1, the flexible shaft 103 is the flexible shaft of a colonoscope, and the device 100 measures the pressure the colonoscope exerts on the bowel 101 of a patient. In other implementations, the flexible shaft may be part of any type of endoscopic device, such as those used for endoscopy surgery of the gastrointestinal tract, respiratory tract, urinary tract, reproductive tract, or other such surgeries. In yet other implementations, the flexible shaft 103 may be part of a medical catheter. In some implementations, the pressure sensing components of device 100 may be included in the non-flexible medical devices. For example, the pressure sensing components described later may be integrated into surgical retractors or forceps in order to indicate to a surgeon the amount of pressure being applied by the medical device to the patient.

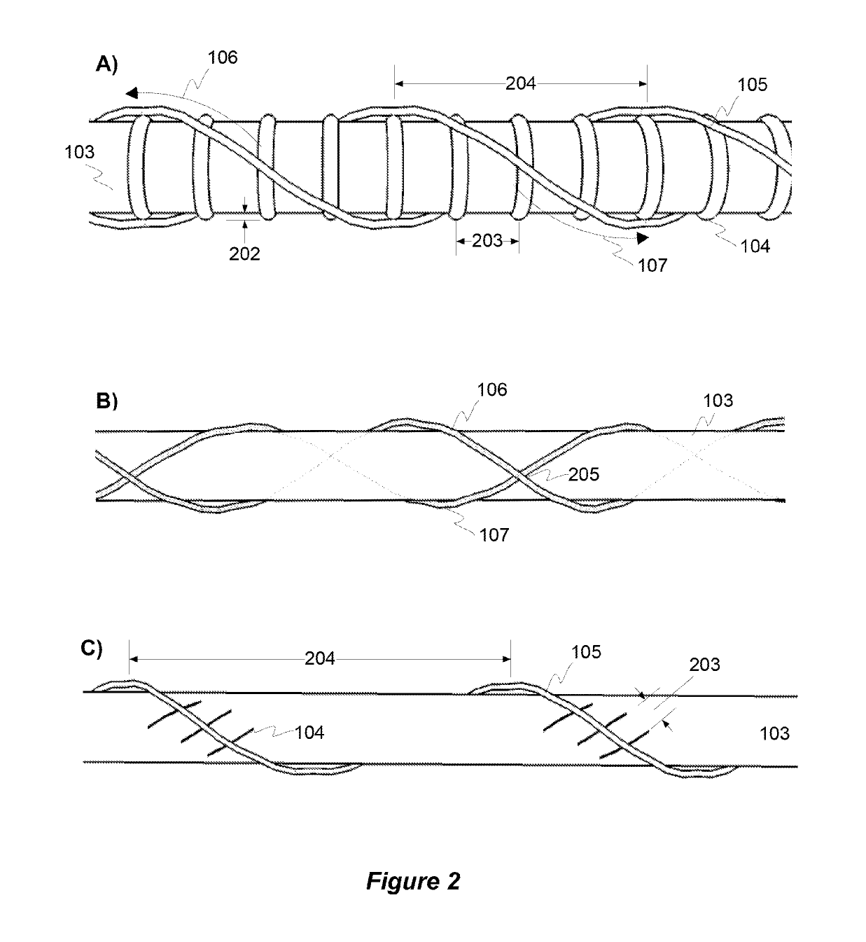

The device 100 may include a plurality of frets 104 along the length of the shaft, discussed in greater detail in relation to FIG. 2 but, briefly, the frets 104 may be raised protrusions spaced along the flexible shaft 103. In some implementations, the frets 104 are spaced equidistant from one another and in other implementations they are spaced non-equidistant from one another. The shape of the fret is not limited to a specific structure. For example, in some implementations the frets 104 are rounded protrusions, while in other implementations the frets 104 may be triangular, frustoconical or square in shape.

The device 100 also includes an optical fiber 105. In some implementations, such as the example illustrated in device 100, the optical fiber 105 is wound from a proximal end of the shaft 103 to the distant end of the shaft 103 and then back to the proximal end of the shaft 103. In other implementations the light source 109 and power meter 108 are placed at opposite ends of the shaft 103, such that the optical fiber 105 only travels from a first end of the shaft 103 to a second end of the shaft 103. In some implementations, the optical fiber 105 is wound such that the outlet portion 107 of the optical fiber does not wrap over the inlet portion 106 of the optical fiber.

In some implementations, the optical fiber 105 is helically wound around the shaft 103. In other implementations, the optical fiber 105 is strung linearly from the proximal end to the distal end of the shaft. For example, the optical fiber may substantially parallel to the length of the shaft 103. In some of these implementations, the flexibility of the optical fiber 105 may be configured to match the tensile properties of the shaft 103 as to not induce artifact into the measurement. In some implementations, the device 100 may include a means to add or remove slack from the optical fiber, such as a spring. Consider the example where two optical fibers run parallel to the shaft 103 and 180 degrees apart from one another. Further consider the shaft 103 is bent such that a first optical fiber 105 runs along the inner diameter of the curved shaft and a second optical fiber 105 runs along the outer diameter of the curved shaft 103. In this example, slack develops in the first optical fiber 105 and a strain develops in the second optical fiber 105. A spring or recoil mechanism would allow the first and second optical fibers to adjust to the flexing shaft 103 without inducing inaccurate pressure readings. In other implementations, the helically wound optical fiber reduces light loss caused by flexing of the shaft 103, by allowing the shaft 103 to flex without inducing pressure on optical fiber 105.

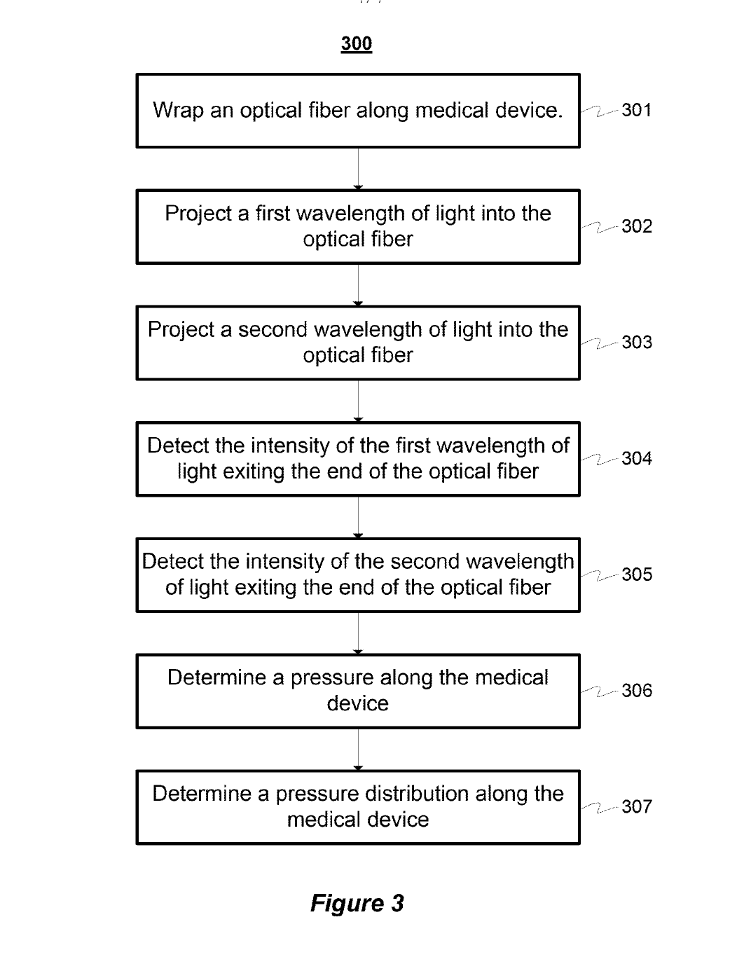

In device 100, the wrapped optical fiber 105 crosses over a plurality of frets 104. When pressure is applied to the device 100, the optical fiber 105 may deform around the fret 104. This small deformation, or microbend, allows a portion of light to escape the optical fiber 105. As discussed below in relation to FIG. 3, the attenuated light can be calculated and associated with a load being applied to the device 100.

In some implementations, the optical fiber 105 is a single-mode optical fiber. Additionally, the optical fiber 105 may be a multi-mode optical fiber. The device 100 may include more than one optical fiber 105. For example, the device may include a second optical fiber wound in tandem with the first optical fiber. In some of these implementations the first and second optical fibers carry light of different wavelengths. In yet other implementations, a plurality of light rays of multiple frequencies may be projected into a single optical fiber. In some implementations, the optical fiber 105 is a fused quartz or glass fiber and in other implementations the optical fiber 105 is a plastic fiber. Discussed further in relation to FIG. 3, but briefly, the use of multiple wavelengths of light allows the pressure distribution of an applied pressure to be calculated.

Still referring to device 100 of FIG. 1, the device also includes a light source 109 connected to the input portion 106 of the optical fiber 105. In some implementations the light source 109 is any source of light. For example, the light source may be, but is not limited to, a light-emitting diode (LED), a fluorescent lamp, a neon lamp, a plasma lamp, a xenon lamp, a laser or other such electrically power light source. In some implementations, the light source emits light including a single or predominate wavelength, multiple predominate wavelengths, or white light. For example, the wavelength of the light may be 850 nm, 1310 nm, or 1550 nm. The device 100 may include filters to restrict broad spectrum light to a specific wavelength. In yet other implementations, the device 100 can include a collimator on the inlet portion 106 of the optic fiber. In some implementations, the light source emits light with a consistent power output. For example, the power output may be a constant value between 0 and −5 dBm. In some implementations, the device 100 includes more than one light sources 109. For example, the device 100 may include a separate light source 109 for each of the optical fibers 105 in a device including more than one optical fiber 105.

The device 100 also includes a power meter 108 connected to the output portion 107 of the optical fiber 105. The power meter 108 measures the light intensity exiting the optical fiber 105 from the outlet portion 107 and provides the processor 110 with the measurement. In some implementations, the power meter 108 has a resolution of 0.1 dB or 0.01 dB and an accuracy of 5%. In other implementations, the intensity of the light exiting the fiber optic 105 at the outlet portion 107 may be measured with a photoresitor, photodiode, or similar optoelectronic device.

As mentioned above, the device 100 also includes a processor 100. In some implementations the device 100 includes more than one processor 110. The processor 110 controls the components of device 100. For example, the processor 110 may control the intensity of light the light source 109 projects into the optical fiber 105. The processor 110 may also receive the readings from the power meters 108 of device 100. The processor 110 may calculate the pressure exerted along the shaft 103 of the device 100. As discussed later, in some implementations, the processor 110 may also calculate the distribution of the pressure. For example, the processor 110 may collect intensity readings from two power meters 108 or one power meter 108 measuring two separate wavelengths to calculate the pressure per unit area. In doing so, the device 100 may determine if an applied pressure is spread along the entire length of the shaft 103 or focalized to a small portion of the shaft 103. The processor may compare the total pressure and/or the pressure per unit area to a set threshold to determine if the pressure being applied by the medical device to the patient is dangerous. The processor 110 may be a microprocessor unit, such as: those manufactured by Intel Corporation of Santa Clara, Calif.; those manufactured by Motorola Corporation of Schaumburg, Ill.; those manufactured by Atmel of San Jose Calif., or any other single- or multi-core processor, or any other processor capable of operating as described herein and performing the calculations described in reference to FIG. 3.

In some implementations, the processor 110 outputs the pressure readings to a display 112. The display may be a screen that provides real-time pressure readings to a user. In other implementations, the display indicates relative pressure measurements. For example, the display may be an indicator light on the handle of the endoscope that is green when the pressure is within safe tolerances, yellow as the pressure approaches the predetermined threshold, and red when the detected pressure exceeds the predetermined threshold. In other implementations, the device may provide a user with audio and/or tactile feedback.

The device 100 is powered by a power source 111. In some implementations, the device 100 is powered by battery power. In other implementations, the device 100 is powered by a medical grade AC power supply.

FIGS. 2A-C illustrate the shaft 103 and wound optical fiber 105 in greater detail. FIGS. 2A-C illustrate non-limiting examples of possible optical fiber 105 winding patterns and fret patterns.

As discussed briefly above in relation to FIG. 1, microbends in an optical fiber may induce significant light attenuation. In some implementations, this causes a portion of the light to be lost to the environment. Additionally, a portion of the light may be backscattered back to the light source. In device 100, a microbend is induced in the optical fiber 105 when the optical is bent over a fret 104 or another portion of the optical fiber 105. As discussed above, wrapping the optical fiber 105 around the shaft 103 minimizes the pressure artifact the shaft 103 induces on the optical fiber 105 when the shaft 103 flexes.

FIG. 2A illustrates, a wrapping pattern similar to the pattern illustrated in device 100 of FIG. 1. In this exemplary implementation, frets 104 are equally spaced along the shaft 103 of the device 100. In other implementations, the frets are not spaced equidistant apart. For example, frets may be spaced more frequently in regions of a device where increased resolution of the pressure detection is required, such as areas of the device more likely to cause injury to a patient. Inversely, frets may be spaced less frequently in areas of the device less likely to come in contact with a patient. In some implementations, the distance 203 between neighboring frets is between 1 mm and 5 mm, between 5 mm and 10 mm, between 10 mm and 20 mm, or between 20 mm and 50 mm. In some implementations, the fret width 201 is less than 1 mm, between 1 and 3 mm, or less than 5 mm. In some implementations, the fret height 202 is less than 1 mm, between 1 and 3 mm, or between 3 and 5 mm.

As described above, and illustrated by device 100, FIG. 2A illustrates a single optical fiber 105 helically wound around the shaft 103 of a device 100. The optical fiber 105 is wound from a proximal end of the shaft 103 to a distal end of the shaft 103. This first portion of the optical fiber 105 is the inlet portion 106. At the distal end of the shaft 103, the optical fiber 105 is helically wound back the original proximal end of the shaft 103. This second portion of the optical fiber 105 is the outlet portion 107. As illustrated in FIG. 2A, the inlet portion 106 of the optical fiber 105 and the outlet portion 107 of the optical fiber 105 do not cross one another. In some implementations, the linear distance 204 it takes the optical fiber 105 to make a 180 degree rotation is between 10 and 20 mm, between 20 mm and 30 mm, or between 30 and 50 mm. This linear distance may also be referred to as the pitch of the optical fiber, In some implementations, the ratio of the linear distance 204 to the fret spacing distance 203 is 4:1. For example, the linear distance 204 may be 36 mm and the fret spacing distance 203 may be 9 mm. In this example configuration, there are 8 sensing axis along the perimeter of the shaft 103, and each of the sensing axis are separated by 45 degrees.

FIG. 2B illustrates a self intersecting optical fiber 105 wrapping configuration. Similar to the above described configuration, the optical fiber 105 in FIG. 2A is helically wound from a proximal end to a distal end and then back to the original proximal end. In some implementations, the device 100 does not have frets 104. In some of these implementations, when the outlet portion 107 of the optical fiber 105 is wound back to proximal end of the shaft 103, it is wound back upon the inlet portion 106. The inlet portion 106 and the outlet portion 107 over lap one another at intersection points 205. The inlet portion 106 and the outlet portion 107 may be wound such that linear distance traveled during a 180 degree rotation is the same. In such an implementation, the device 100 would have two sensing axis. In some implementations, the number of sensing axis may be increased by decreasing the linear distance traveled during a 180 degree rotation of one of the portions of the optical fiber 105. In other implementations, the self-intersecting optical fiber design also includes frets.

FIG. 2C illustrates a third optical fiber 105 wrapping configuration. In this configuration, an optical fiber 105 is wound from a proximal end of the shaft 103 to a distal end of the shaft 103 and back to the proximal end of the shaft 103. Similar to the configuration illustrated in FIG. 2A, the inlet portion 106 and the outlet portion 107 of the optical fiber do not intersect. In some implementations, as illustrated in FIG. 2C, the frets 104 only partially circumnavigate the circumference of the shaft 103. In some implementations, the frets 104 are configured such that each fret 104 is perpendicular to the optical fiber 105. In other implementations, the fret may be non-perpendicular to the optical fiber 105. For example, the fret 104 and optical fiber 105 could intersect one another at 30 degrees. This partial fret configuration may have similar linear distances 204 and fret spacing distances 203 as described above.

In some implementations of the above configurations, the fret spacing 203 and the optical fiber spacing 204 are adjustable. For example, the device 100 may be configured such that a surgeon may rearrange the frets 104 and optical fiber 105 to provide greater pressure distribution resolution. Additionally, in some implementations, more than one optical fiber 105 is wound around the shaft 103 of the device 100. For example, a second optical fiber may be wound in tandem with the first optical fiber or a first optical fiber 105 may be wound from the proximal end to the distal end and then a second fiber optic 105 may be wound from the distal to the proximal end. In some implementations, these multiple optical fibers 105 have separate light sources and in some implementations they share a single light source.

Referring now to FIG. 3, illustrated is a flow chart of a method 300 for determining the pressure a medical device, similar to the device of FIG. 1, induces on a patient. First, an optical fiber is wrapped along the shaft of a medical device (step 301). A first wavelength of light is projected into the optical fiber (step 302) and a second wavelength of light is projected into the optical fiber (step 303). Responsive to projecting the wavelengths of light into the optical fiber, the intensity of the first wavelength of light exiting the optical fiber is detected (step 304) and the intensity of the second wavelength of light exiting the optical fiber is detected (step 305). Responsive to detecting the intensities of the wavelengths of light exiting the optical fiber, a pressure along the medical device is calculated (step 306) and a pressure distribution along the medical device is determined (step 307).

As set forth above, and referring to FIG. 1, the method of detecting a pressure includes wrapping an optical fiber along the shaft of a medical device (step 301). As described above, at least one optical fiber is wrapped from a proximal end of a medical device to a distal end of the medical device and then back to the proximal end. In some implementations, the optical fiber is wrapped along a shaft that includes a number of frets. In some implementations, the fret and optical fiber assembly is removable from the medical device and in other implementations it is permanently attached to the medical device. For example, the optical fiber and fret assembly can be built into a sleeve which is not permanently slid over the shaft of an endoscope. The optical fiber and frets assembly may be disposable and discarded after a single use.

A first wavelength is projected into a first optical fiber (step 302). As described above, in some implementations, the light source may be a light lamp, a laser, or a LED. In some implementations, the wavelength of the first wavelength is 1550 nm. In some implementations, a second wavelength of light is projected into the optical fiber (step 303). In some implementations, the second wave of light is projected into a second optical fiber. The wavelength of the second wavelength may be 1310 nm.

The method 300 continues with the detection of the intensity of the first wavelength as it exits the optical fiber (step 304) and the detection of the intensity of the second wavelength of light as it exits the optical fiber (step 305). As described above, a portion of light may be lost to the environment at each microbend of the optical fiber. Accordingly, if a pressure causes the optical fiber to bend around a fret, the intensity of the light exiting the optical fiber will be less the optical fiber entering the optical fiber.

Responsive to detecting the intensities of the first and second wavelength of light exiting the optical fiber, the device determines the pressure asserted along the device (step 306). In some implementations, the pressure along the device is determined by using only the detected intensity of the first wavelength of light exciting the optical fiber. In these implementations, a known initial power (Pᵢ) of the first wavelength of light is projected into the optical fiber. The power meter determines the exiting power of the first wavelength of light (P_{f}). In some implementations, a portion of Pᵢ will be lost when the optical fiber bends around a fret. This light attenuation (ΔP=Pi−P_{f}) can be used to calculate the bend radius of the optical fiber, which in turn can be used to determine the pressure applied to the optical fiber. In one embodiment, the processor first calculates the an amplitude loss coefficient (2α) using the equation:

$$Δ P = - 10 {log}_{10} ( \frac{1}{1 + 2 α} ) \qquad Equation 1$$

After calculating 2α, the bend radius (R) can be calculated with:

$$2 α = \frac{\sqrt{π} {K}^{2} exp [ - \frac{2}{3} ( \frac{{γ}^{3}}{{β}_{g}^{2}} ) R ]}{{e}_{V} {γ}^{1.5} {V}^{2} \sqrt{R} {K}_{V - 1} ( γα ) {K}_{V + 1} ( γα )} \qquad Equation 2$$

In Equation 2, K is the field decay rate in the fiber core, β_{g} is the propagation constant, γ is the field decay in the fiber cladding, eᵥ is a constant that depends on the propagation mode in the fiber, and V is the propagation factor.

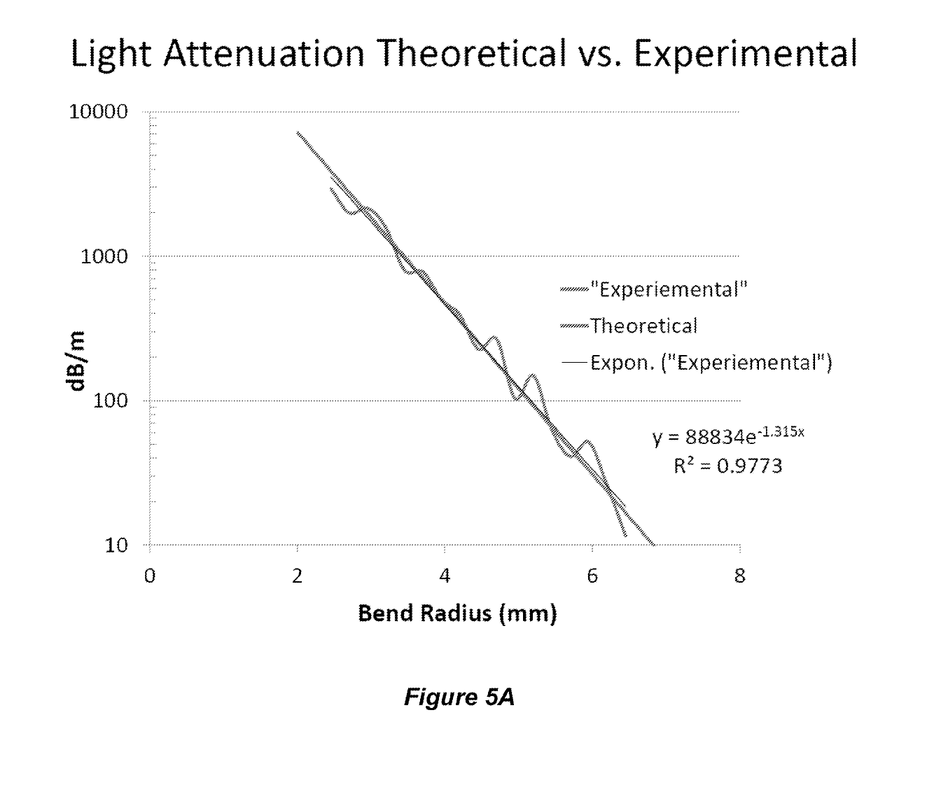

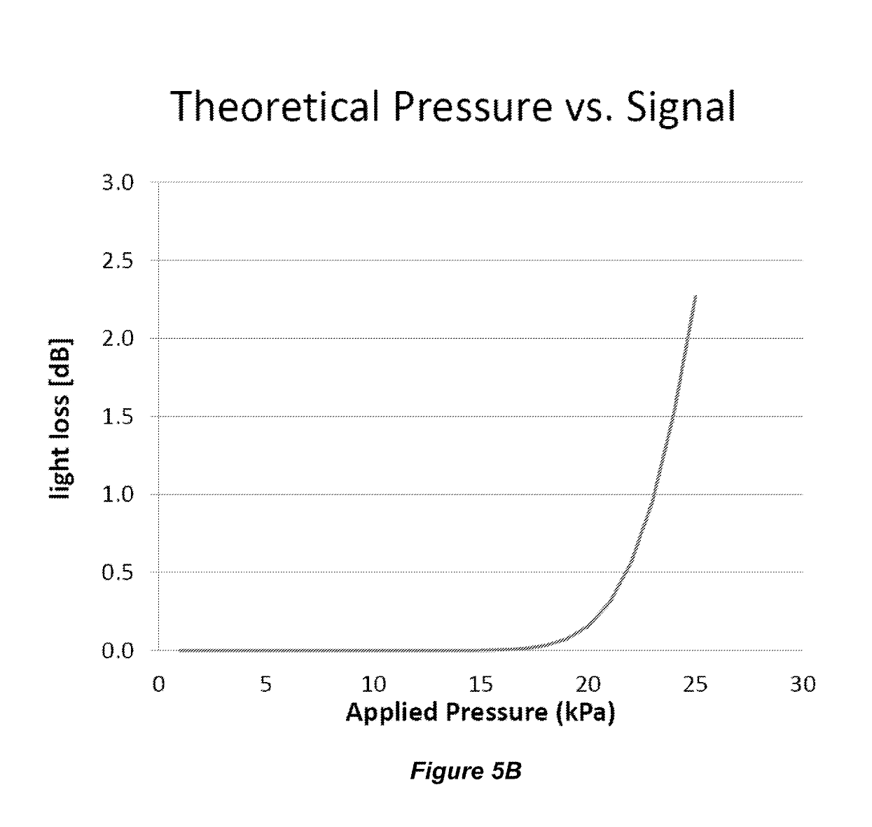

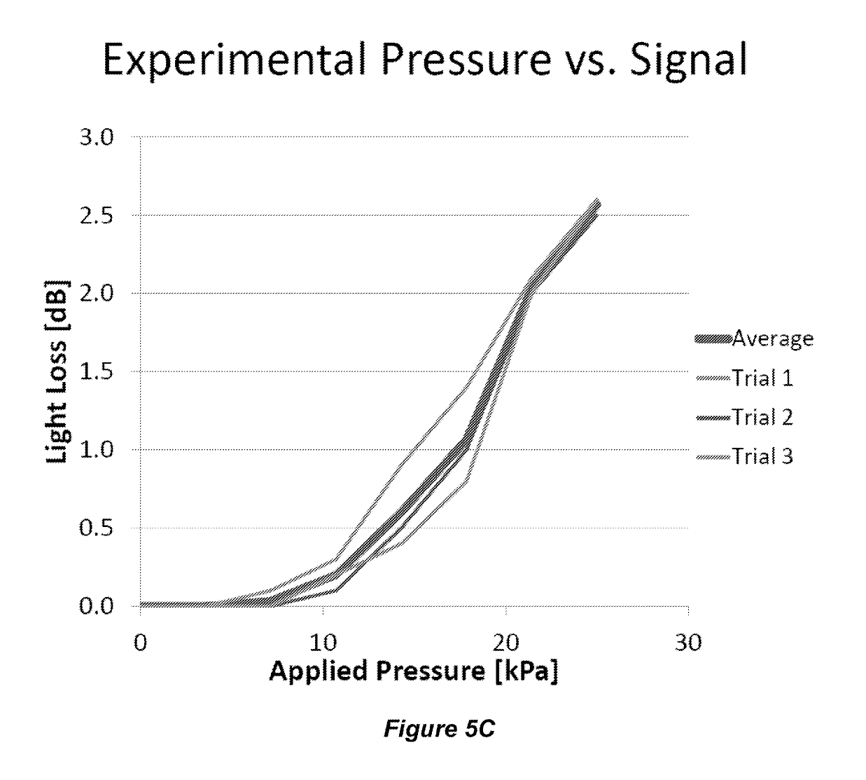

FIG. 5A illustrates the light attenuation vs. different bend radiuses. FIG. 5A illustrates there is a linear relationship between the bend radius and light attenuation when the light attenuation is plotted on a logarithmic scale. Furthermore, FIG. 5A shows the experimental results from applying a pressure to a device similar to device 100 are highly correlated with the theoretical values using the equations described herein.

Next, modeling the optical fiber as a beam with fixed ends experiencing a uniformly distributed load (w), the load can be calculated with:

$$w = \frac{384 REI}{l} \qquad Equation 3$$

In equation 3, E is the modulus of elasticity, I is the moment of inertia, and l is the width of the fret. R is the calculated bend radius from Equation 2. In some implementations, these calculations are continually calculated by the processor or calculated at a given interval. In other implementations, the above equations are used to generate a lookup table that the processor uses to associate a given light loss with an applied pressure. In yet other implementations, when graphed on a log scale, the relationship between light loss and applied pressure is modeled with a linear equation that allows for a direct, approximate calculation of the applied pressure.

FIG. 5B-C illustrate the relationship between the applied pressure and the light loss from the optical fiber. FIG. 5B illustrates the theoretical relationship between the applied pressure and the light loss, and FIG. 5C illustrates experimental results using a device similar to device 100 of the relationship between applied pressure and light loss. Again, the experimental results are highly correlated to the theoretical values. Additionally, FIG. 5C illustrates the device is highly sensitive to pressure changes in the range of 10 to 30 kPa, a key pressure range experienced in bowel perforations.

Again referring to method 300, the method continues by calculating a pressure distribution along the medical device. In some implementations, the light loss when the optical fiber bends over multiple frets is additive. For example, it may be difficult to delineate 10 kPa applied to two frets from 20 kPa applied to one fret. As described above, in some implementations, a second wavelength of light is projected into the optical fiber. The second wavelength of light may be used as a reference wavelength. The second wavelength of light may be projected down the same optical fiber as the first wavelength of light or it may be projected down a second optical fiber. The reference wavelength of light may be near the first wavelength of light. In some implementations, the second wavelength of light is selected such that it reacts differently to applied pressures and microbends. For example, 1550 nm may be chosen as the first wavelength and 1310 may be chosen as the second wavelength. In some implementations, the second wavelength of light is chosen such that it is less sensitive to microbends. For example, the first wavelength of light may experience attenuation starting at 10 kPa, while the second wavelength may experience attenuation starting at 20 kPa. By taking the ratio of the detected power of the first wavelength of light to the detected power of the second wavelength of light, the processor can distinguish high pressures that are distributed over a large area and are therefore not dangerous from high pressures distributed over a small area that are dangerous. For example and referring to FIG. 5C, the processor may detect a 2.0 dB light attenuation in the first wavelength, and the processor may detect no light attenuation in the second wavelength of light. Not represented in FIG. 5C, but the second wavelength of light may have a comparable pressure vs. attenuation curve as shown in FIG. 5C; however, the curve of the second wavelength would be shifted to the right. By comparing the attenuation values of the first and second wavelengths, the processor can determine the 2.0 dB light attenuation was caused by multiple microbends at values less than about 20 kPa rather than a single microbend caused by a 20 kPa pressure.

What is claimed:

1. A pressure sensing medical device comprising:
an elongated shaft having a first end, a second end, and a plurality of frets spaced a set distance apart along a substantial portion the elongated shaft;
a first optical fiber having an inlet and an outlet and running along a substantial length of the elongated shaft;
a first light source configured to project light into the inlet of the first optical fiber; and
a first sensor positioned to detect light emitted from the outlet of the first optical fiber.

2. The device of claim 1, wherein the first light source and the first sensor are position near the first end of the elongated shaft and connected by the first optical fiber.

3. The device of claim 1, wherein the first optical fibers run non-tangentially over the frets.

4. The device of claim 1, wherein the medical device further comprises a processor configured to calculate a pressure exerted on the elongated shaft.

5. The device of claim 1, wherein the first optical fiber is helically wound around the elongated shaft.

6. The device of claim 5, wherein the pitch of the first helically wound optical fibers is four times the set distance between adjacent frets.

7. The device of claim 1, wherein the first light source is configured to project light of a first wavelength and second wavelength.

8. The device of claim 7, wherein the first wavelength and second wavelength are selected attenuate at different rates when the first optical fiber is bent a set amount.

9. The device of claim 7, wherein the first optical fiber is multi-modal.

10. The device of claim 1, wherein a second optical fiber is helically wrapped along the elongated shaft substantially parallel to the first optical fiber.

11. The device of claim 10, wherein the second optical fiber is connected to a second sensor and a second light source emitting a second wavelength of light.

12. The device of claim 1, wherein the elongated shaft is part of a catheter, an endoscope, or a colonoscope.

13. A method for measuring a pressure along a medical device, the method comprising:
wrapping a first optical fiber along an elongated shaft of the medical device, wherein a plurality of frets are spaced a set distance apart long substantial portion of the elongated shaft;
projecting, by a light source, a first wavelength of light into the first optical fiber, wherein the first wavelength of light has a first intensity;
detecting, by a sensor, a second intensity of the first wavelength of light when the first wavelength of light exits the optical fiber; and
determining, by a processor, a pressure along the elongated shaft of the medical device by comparing the first intensity to the second intensity.

14. The method of claim 13, further comprising:
projecting, by the light source, a second wavelength of light into the first optical fiber, wherein the second wavelength of light has a third intensity; and
detecting, by the sensor, a fourth intensity of the second wavelength of light when the second wavelength of light exits the optical fiber; and
determining, by the processor, a distribution of the pressure along the elongated shaft of the medical device by comparing a first difference between the first intensity and second intensity to a second difference between the third intensity and fourth intensity.

15. The method of claim 14, wherein the first wavelength and second wavelength are selected to attenuate at different rates when the first optical fiber is bent a set amount.

16. The method of claim 13, further comprising:
wrapping a second optical fiber along the elongated shaft of the medical device;
projecting, by a second light source, a second wavelength of light into the second optical fiber, wherein the second wavelength of light has a third intensity; and
detecting, by a second sensor, a fourth intensity when the second wavelength of light exits the second optical fiber; and
determining, by the processor, a distribution of the pressure along the elongated shaft of the medical device by comparing a first difference between the first intensity and second intensity to a second difference between the third intensity and fourth intensity.

17. The method of claim 13, further comprising warning a user if the pressure exceeds a set threshold.

18. The method of claim 13, further comprising wrapping the first optical fiber around the length of the elongated shaft and non-tangentially over the frets.

19. The method of claim 13, further comprising wrapping the first optical fiber helically around the elongated shaft with a first pitch.

20. The method of claim 19, wherein the first pitch of the first helically wound optical fiber is four times the set distance between adjacent frets.
